(19) United States
(12) Patent Application Publication
(10) Pub. No.: US 2009/0069542 A1
(43) Pub. Date: Mar. 12, 2009

(54) Process Of Extracting High Quality Proteins From Cereal Grains And Their ByProducts Using Acidic Medium And A Reducing Agent

(75) Inventors: Yiqi Yang, Lincoln, NE (US); Weijie XU, Lincoln, NE (US); Narenda Reddy, Lincoln, NE (US)

Correspondence Address: THOMPSON COBURN, LLP, ONE US BANK PLAZA, SUITE 3500, ST LOUIS, MO, 63101, US

(73) Assignee: BOARD OF REGENTS OF THE UNIVERSITY OF NEBRASKA, Lincoln, NE (US)

(21) Appl. No.: 11/862,339

(22) Filed: Sep. 27, 2007

Related U.S. Application Data
(60) Provisional application No. 60/971,728, filed on Sep. 12, 2007.

(51) Int. Cl. C07K 14/415; C07K 14/425

(52) U.S. Cl. 530/373; 530/370; 530/372; 530/376; 530/375; 530/374; 530/377; 530/422; 530/417

(57) ABSTRACT

The present invention is directed to a method for processing a plant-based protein source, the method comprising an acidic extracting solution comprising a reducing agent is useful for extracting and isolating proteins from plant-based protein sources.

### CROSS-REFERENCE TO RELATED APPLICATIONS

This application is claim priority to U.S. Provisional Patent Application No. 60/971,728, filed on Sep. 12, 2007, which is hereby incorporated by reference herein in its entirety.

### BACKGROUND OF THE INVENTION

1. Field of the Invention

The present invention relates to a method of extracting protein from a plant-based protein source such as cereal grains and byproducts of cereal grain processing operations.

2. Description of the Related Technology

Cereal grains are an abundant and renewable source of plant-based proteins. Proteins derived from cereal grains tend to have certain properties (e.g., insolubility in water, mechanical stability, thermal stability, etc.) that can be useful for commercial and industrial applications. Products comprising plant-based proteins include fibers, films, paints, and adhesives. For example, the main protein present in corn is the protein zein. “Zein,” as used herein, refers to a heterogeneous mixture of prolamine proteins present in corn that may be extracted from corn or the byproducts produced by the processing of corn. Zein is a potential useful protein for several applications because of its relatively low hydrophilicity, elasticity, and film-forming capabilities (Dickey et al., “Zein batch extraction from dry-milled corn: cereal disintegration by dissolving fluid shear.” Cereal Chem. 1998, 75, 433-448). Because of such advantageous properties, zein has been incorporated into products such as fibers, films, and adhesives (Lawton, “Zein: a history of processing and use.” Cereal Chem. 2002, 79, 1-18; Yang et al. “Formaldehyde-free zein fiber: preparation and investigation.” J. Appl. Polym. Sci. 1996, 59, 433-441; Fu et al. “Zein: Properties, Preparations, and Applications.” Food Sci. Biotechnol. 1999, 8, 1-10; Severinghaus, J. “Where will all the DDGS go?” Iowa Farm Bur. Int. Trade Analyst 2006, 4, 1-2; Loy and Wright, “Nutritional properties and feeding value of corn and its by-products.” In Corn Chemistry and Technology, 2nd ed.; White, P. J., Johnson, L. A., Eds.; American Association of Cereal Chemists: St. Paul, Minn., 2003; pp 591). Thus, it would be useful to develop processes for the extraction of zein and other cereal grain proteins to be used for such applications.

Cereal grain proteins may be extracted from cereal grains or from the byproducts of the processing of cereal grains. Extraction of protein from grains that have not been ground or processed may be difficult, especially if the protein is inaccessible to extraction. For instance, storage proteins encased within the seed coat typically require milling to expose them to extraction conditions. More typically, however, cereal grain proteins are extracted from the byproducts of cereal grain processing. Because methods of processing cereal grains involve the extraction or use of fractions other than the protein fraction, the byproducts of cereal grain processing are a potential source of plant protein. Additionally, further processing of byproducts reduces waste and the need for the disposal of the byproducts. Another advantage is that the byproducts of processed grains are typically available at lower cost than whole or unprocessed grains.

Cereal grain byproducts that may be used as sources of cereal grain proteins include gluten meal and dried distillers grains. “Gluten meal”, as used herein, refers to the byproduct of the wet-milling of cereal grains. Methods to extract proteins from corn gluten meal are disclosed for instance in U.S. Pat. Nos. 3,535,305; 2,733,234; 2,414,195; 2,332,356; 2,105,760; 2,206,819; 5,254,673; 6,602,985; and Parris and Dickey, “Extraction and solubility characteristics of zein proteins from dry-milled corn.” J. Agric. Food Chem. 2001, 49, 3757-3760. These previous methods have all used either aqueous alcohols under alkaline conditions or alkaline solutions to extract proteins. These methods typically have a low yield of proteins and/or the extracted proteins are of lesser quality (e.g., low molecular weight and viscosity).

Typically, commercial zein is extracted from corn gluten meal at a reported cost of about $8-10 per pound. Methods to obtain zein at lower cost from dried distillers grains with solubles (DDGS) have been attempted because DDGS is typically available for a significantly less cost than gluten meal. “Dried distillers grains” or “DDG”, as used herein, refer to the solids remaining after fermentation of cereal grains and distillation of the alcohol produced by the fermentation of cereal grains. “Dried distillers grains with solubles” or “DDGS”, as used herein, refer to dried distillers grains with soluble components, including cereal proteins and fats. Because DDGS is rich in protein, fat, and crude fiber (29.7, 8.8, and 9.3% based on dry weight, respectively), it is a potential source of raw material for producing valuable products (Loy, D. D.; Wright, K. N. Nutritional properties and feeding value of corn and its by-products. In Corn Chemistry and Technology, 2nd ed.; White, P. J., Johnson, L. A., Eds.; American Association of Cereal Chemists: St. Paul, Minn., 2003; pp 591).

Distillers dried grains may provide an abundant source of cereal grain protein because of the current demand for ethanol to be used as a fuel source. The increasing price of petroleum products has encouraged the development and production of alternative fuel sources, including ethanol and biodiesel. Currently, about 20% of the corn produced in the United States is used to produce about 5 billion gallons of ethanol every year. Thus, the production of ethanol from the processing of corn is expected to generate about 10 million tons of DDGS a year (Severinghaus, “Where will all the DDGS go?” Iowa Farm Bur. Int. Trade Analyst 2006, 4, 1-2). Currently, DDGS has limited uses in food and feed applications and as a result it is not unusual for it to be discarded.

Previous methods have also included reducing agents in alkaline solution in an attempt to increase the yield of proteins extracted from cereal grain byproducts (Wolf and Lawton, “Isolation and characterization of zein from corn distiller's grains and related fractions.” Cereal Chem. 1997, 74, 530-536; Parris and Dickey, “Extraction and solubility characteristics of zein proteins from dry-milled corn.” J. Agric. Food Chem. 2001, 49, 3757-3760; Shukla and Cheryan, “Zein: the industrial protein from corn.” Ind. Crops Prod. 2001, 13, 171-192; and Dickey et al. “Ethanolic extraction of zein from maize. Ind. Crops Prod. 2001, 13, 67-76; 11. Wu et al., “Protein-rich residue from corn alcohol distillation: fractionation and characterization.” Cereal Chem. 1981, 58, 343-346). Wolf and Lawton reported using a reducing agent for zein and other protein extraction from dried distillers grains and other corn distillation fractions by using 0.1M sodium hydroxide, 0.1% dithiothreitol (DTT) and 0.5% sodium dodecyl sulfate for 30 minutes. Zein was extracted from this crude protein using 60% ethanol at 60° C. About 1.5-6.6% crude zein based on the total weight of the DDGS was obtained, but the proteins had low purity of 37-57%. Except for the yield and the composition of the proteins obtained, this study did not report on properties (e.g., the molecular weight, viscosity, etc.) which may affect the quality of the zein obtained for applications. In comparing the extraction with and without the addition of DTT, researchers have concluded that it is necessary to use a reducing agent to obtain high yields of zein. (Wolf and Lawton, “Isolation and characterization of zein from corn distiller's grains and related fractions.” Cereal Chem. 1997, 74, 530-536; Shukla and Cheryan. “Zein: the industrial protein from corn.” Ind. Crops Prod. 2001, 13, 171-192; Wu et al., “Protein-rich residue from corn alcohol distillation: fractionation and characterization,” Cereal Chem. 1981, 58, 343-346.

Although many of the foregoing methods of extracting proteins from plant-based protein sources have been effective to various degrees, a need continues to exist for a method of extracting high-quality proteins from protein plant-based sources at high yield and low cost. It would be desirable to find uses for byproducts from cereal grain to eliminate waste and the need for disposal of byproducts. It would also be desirable to use the byproducts from the processing of cereal grains in order to lower the cost of proteins obtained by extraction. Obtaining high-quality plant-based proteins at low cost is expected to make plant-based proteins a useful source for fiber, film, adhesive, and other applications. The increasing availability of DDGS as byproduct of ethanol production could help to obtain zein at low cost and facilitate the development of industrial applications for zein. Conversely, developing industrial applications with large market and high value addition for DDGS and zein would also help to decrease the cost of ethanol production. Fibrous applications offer both the large market and high value addition for the proteins and other products obtained from DDGS.

### BRIEF SUMMARY OF THE INVENTION

Briefly, therefore, the present invention is directed to a method for processing a plant-based protein source, the method comprising contacting the plant-based protein source with a protein extraction fluid to dissolve protein from the plant-based protein source into the protein extraction fluid and isolating the dissolved protein from the protein extraction fluid, wherein the protein extraction fluid comprises a protein reducing component for breaking disulfide bonds between proteins and has a pH that is no greater than about 8.

Additionally, the present invention is directed to a method of separating oil, pigment, and protein from a cereal grain material, the method comprising: performing a first treatment on the cereal grain material, the first treatment comprising contacting the cereal grain material with an anhydrous alcohol to form a first mixture having a liquid-to-solid weight ratio of at least about 1:1 and no greater than about 1000:1 and a temperature of at least about 10° C. and no greater than the boiling point of the anhydrous alcohol for a duration sufficient to remove substantially all of the oil, pigment, or both from the cereal grain material; separating the first mixture into a first solids portion comprising the cereal grain material and a first liquid portion comprising anhydrous ethanol and dissolved oil, pigment, or both; performing a second treatment on the first solids portion, the second treatment comprising contacting the cereal grain material with an acidic aqueous alcohol solution that comprises a protein reducing agent for breaking disulfide bonds between proteins to form a second mixture having a liquid-to-solid ratio of at least about 1:1 and no greater than about 1000:1 and a temperature of at least about freezing point and no greater than about the boiling point of the acidic alcohol solution for a duration that is at least about 10 minutes and no greater than about 24 hours; and separating the second mixture into a second solids portion comprising the cereal grain material and a second liquid portion comprising the acidic alcohol solution and dissolved protein.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 contains the FTIR spectrums of oil extracted from DDGS and a commercially available corn oil.

FIG. 2 contains the FTIR spectrums of solids obtained with oil extracted from DDGS and commercially available zein.

FIG. 3 shows the yield of zein (% based on the dry weight of DDGS used) using protein extraction fluids of the present invention having differing pH values (70% (w/w) ethanol with 0.25% sodium sulfite at boil for two hours with a solvent to solids ratio of 10:1).

FIG. 4 shows the yield of zein (% based on the dry weight of DDGS used) using protein extraction fluids of the present invention having differing concentrations of sodium sulfite (70% (w/w) ethanol at boil for two hours at a pH of 2 and a solvent to solids ratio of 10:1).

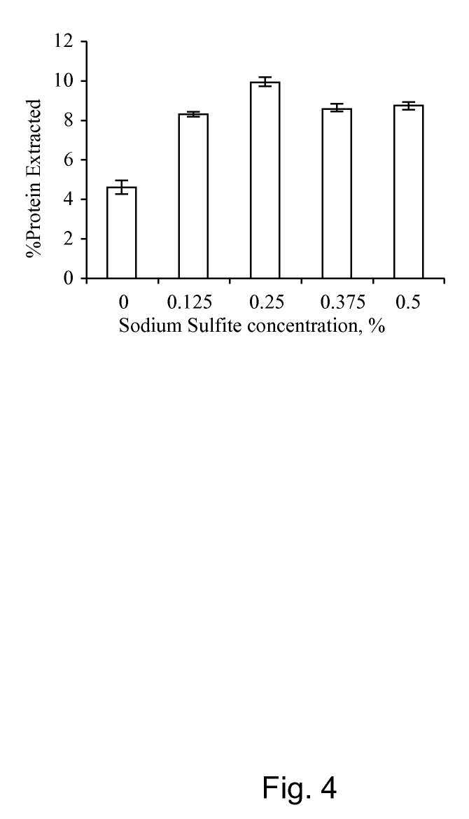

FIG. 5 shows the yield of zein (% based on the dry weight of DDGS used) when extracted using a protein extraction fluid of the present invention at differing temperatures (70% (w/w) ethanol and a sodium sulfite concentration of 0.25% for 2 h and solvent to solids ratio of 10:1 and a pH of 2).

FIG. 6 shows the intrinsic viscosity of the zein using a protein extraction fluid of the present invention maintained at differing temperatures (70% (w/w) ethanol and a sodium sulfite concentration of 0.25% for two hours and solvent to solids ratio of 10:1 and a pH of 2).

FIG. 7 shows the yield of zein (% based on the dry weight of DDGS used) using a protein extraction fluid of the present invention for differing durations (70% (w/w) ethanol at boil with a solvent to solids ratio of 10:1 and sodium sulfite concentration of 0.25% at pH 2).

FIG. 8 shows the yield of zein (% based on the dry weight of DDGS used) using a protein extraction fluid of the present invention at differing solvent to solids ratios (70% (w/w) ethanol at boil for two hours and sodium sulfite concentration of 0.25% at pH 2).

FIG. 9 contains the FTIR spectrums of crude zein obtained using a protein extraction fluid of the present invention having a pH of about 2 (the FTIR spectrums of zein obtained at other pH conditions studied were similar) and a commercially available zein.

FIG. 10 contains the SDS-PAGE of proteins extracted from DDGS and commercial zein. Lanes 1 and 3 are commercial zein in reduced and unreduced forms, respectively. Lanes 2 and 4 are proteins from DDGS in reduced and unreduced forms, respectively.

### DETAILED DESCRIPTION OF THE INVENTION

In accordance with the present invention, it has been discovered that, an acidic extraction solution comprising a reducing agent is useful for extracting and isolating proteins from plant-based protein sources. Thus, in one embodiment, the present invention is directed to a method for processing a plant-based protein source, wherein proteins are extracted from a plant-based protein source using a protein extraction fluid that comprises a protein reducing component and has an acidic pH. Additionally, the method of extracting protein of present invention may also be accompanied by a method(s) for extracting oil and pigments from the plant-based protein source before extraction of proteins. The extraction of oils and/or pigments is optional and is not required in order to extract protein from the plant-based protein source. The separation of oils, pigments, and other impurities from any fractions comprising proteins may be performed before or after extraction of the protein. That said, it is to be noted that the conditions for extracting proteins may require modification to optimize the protein extraction if the oil and pigment are to be extracted after extraction of the proteins. Typically, it is preferred to separate any extracted oil, pigments, and/or other impurities from the plant-based protein source before performing the protein extraction process because they may reduce the total amount of protein extracted and affect the quality of the protein extracted (e.g., as determined by color, purity, molecular weight, viscosity, etc.). If protein is extracted in the presence of oils, pigments, and/or impurities extra extraction processes and/or more vigorous conditions may be used and/or subsequent purification processes may be implemented.

In the method of the invention it is possible to use any plant-based protein source or combination of sources. Such sources include cereal grains and byproducts from the process of cereal grains. When available, performing the process of the present invention on a plant-based protein source that includes at least some portion of cereal grain byproducts is typically desirable because they tend to cost less than whole cereal grains. Additionally, depending upon the particular circumstances (e.g., the desire properties of the extracted protein), it may be advantageous for the plant-based protein source to be comprised primarily of byproducts and possibly even entirely of byproducts. Any cereal grain byproduct that contains protein may be used. Examples include gluten meal, gluten feed, and dried distillers grains. Gluten meal is produced by wet-grinding of cereal grains. Appropriate gluten meals include, for example, those made from corn, wheat, sorghum, barley, soybean, canola, soybean, oat, rice, and any combination thereof. Among cereal byproducts, DDGS may be preferred because it typically costs the least. As mentioned above, DDGS is produced by processing cereal grains such as corn, wheat, sorghum, barley, rye, and combinations thereof for fermentation and distillation. Proteins that are present in DDGS and may be extracted from DDGS include major seed storage proteins, such as zein, glutelin, and gliadin. The plant-based protein source may need to be ground to expose the proteins to the protein extraction fluid. Typically, the plant-based protein source is ground so that it can pass through a sieve having openings of 850 micrometers (20 mesh).

As mentioned above, the plant-based protein source may comprise an oil, a pigment, or both. Such a plant-based protein source may be treated to extract oil and pigments before protein extraction. For example, the plant-based protein source may be contacted with an oil-pigment extraction fluid to dissolve oil, pigment, or both from the plant-based protein source into the oil-pigment extraction fluid before contacting the plant-based protein source with the protein extraction fluid. The oil-pigment extraction fluid may comprise an alcohol, benzene, hexane or another organic solvent, or any combination of the foregoing that is capable of dissolving the oil and/or pigment from the plant-based protein source. In general, it is desirable to limit the amount of water in the oil-pigment extraction fluid so as to limit the amount of proteins that may be dissolved therein. As such, it is preferred that the oil-pigment extraction fluid comprises no more than 5% w/w of water. More preferably, the oil-pigment extraction fluid is essentially free of water. In view of the foregoing, it is generally desirable for the oil-pigment extraction fluid to be anhydrous ethanol because it is readily attained, relatively inexpensive, and most, if not all, of the plant oils and pigments are quite soluble in ethanol.

The oil, pigment, or both dissolved in the oil-pigment extraction fluid may be isolated from the oil-pigment extraction fluid. The oil-pigment extraction fluid may also dissolve proteins from the plant-based protein source and the dissolved proteins may be isolated from the oil-pigment extraction fluid. In general, the pretreatment of the plant-based protein source comprises contacting the cereal grain material with the oil-pigment extraction fluid by any appropriate method such as by spraying, soaking, immersion, etc. without our without agitation of solid material. To date, acceptable results have been obtained by contacting the solids with the oil-pigment extraction fluid in a manner so that a mixture having a liquid-to-solid weight ratio of at least about 1:1 and no greater than about 1000:1 is formed. The mixture is typically kept within the temperature range of at least about 10° C. and no greater than the boiling point of the oil-pigment extraction fluid. Also, the solids and liquid are typically in contact for a duration to sufficient to remove at least some of the oil and/or pigment from the plant-based protein source. Preferably, the duration of the contact is at least sufficient to remove substantially all of the oil, pigment, or both from the cereal grain material. After completion of the oil-pigment extraction operation, a solids portion comprising the cereal grain material and a liquid portion comprising the oil-pigment extraction fluid and dissolved oil, pigment, or both are separated from each other by any appropriate method such as filtration. The dissolved oil, pigment, and/or any dissolved protein may be separated from the liquid portion by any appropriate method such as evaporation, filtration, centrifugation, chromatography, or any combination thereof. The solids portion is subjected to a protein extraction operation.

The protein extraction operation comprises contacting a plant-based protein source, which may have been subjected to the aforementioned oil-pigment extraction process, with a protein extraction fluid comprising as organic solvent component capable of solubilizinng proteins in the source material. Appropriate organic solvent components include, for example, alcohols, alkalies (e.g., sodium hydroxide), ketones, amines (e.g., C₆H₅N), amides (e.g., acetamide), acids (e.g., hydrochloric acid), and combinations thereof. Alcohols, in general, and ethanol, in particular, are typically preferred over other organic solvents for the same reasons set forth above and it is convenient for ethanol producing companies to extract cereal grain proteins using ethanol because DDGS is a byproduct of ethanol production.

Typically, the organic solvent component constitutes at least about 5% by weight of the protein extraction fluid. The organic solvent component may comprise up to 100% of the protein extraction fluid but typically it constitutes no more than about 70% by weight of the protein extraction fluid. The protein extraction fluid may also comprise water. The concentration of water in the protein extraction fluid may be as high as 95% by weight or even greater but typically it is no greater than about 30% by weight of the protein extraction fluid. The concentrations of the solvent and water, if present, most effective for extraction of a particular protein or class of proteins may be empirically determined by methods known to one of ordinary skill in the art. For example, zein does not dissolve in 100% alcohol and it has been determined that a 70% aqueous ethanol solution provides the optimal solubility under certain conditions (Zein properties and alternative recovery methods (dissertation) by Fu, Dejing, Ph.D., The University of Nebraska—Lincoln, 2000, 188 pages; AAT 9967416).

The protein extraction fluid further comprises a pH adjusting component. Suitable pH adjusting components include hydrochloric acid, sodium hydroxide, sodium carbonate, acetic acid, inorganic acids, organic acids, and combinations thereof. The pH of the protein extraction solution may be adjusted prior contacting the fluid with the protein source and may be adjusted or monitored in subsequently during the extraction process. That said, references herein to the pH of the protein extraction fluid are to the pH of the protein extraction fluid before the inclusion of components in addition to the aforementioned organic solvent component and optional water and before being contacted with the protein source. For example, the pH usually changes after inclusion of a reducing agent in the protein extraction fluid. In particular, adding sodium sulfite as a reducing agent will typically increase the pH. Also, the pH of a solution typically changes with temperature. A person of skill in the art will be able to determine the pH most effective for extracting a particular protein. One method for determining an optimal pH is based on the isoelectric point (pH at which a molecule or surface carries no net electrical charge) of the protein being extracted, which is believed to have its lowest solubility at or about its isoelectric point. Increasing or decreasing the pH increases the net negative or positive charges on the protein, respectively, tends to result in higher protein solubility.

Regarding the pH of the protein extraction fluid, it is believed that acidic conditions tend to promote solubility of the proteins in the protein source by breaking chemical linkages formed between the protein to be extracted and other materials in the plant-based protein source, especially at temperatures above 50° C. In particular, without being held to a particular theory, it is believed that ester or ether linkages that may exist between the carboxyl or hydroxyl groups in the proteins and the hydroxyl groups in the polysaccharides (cellulose, hemicellulose, and starch) of cereal grain byproducts are more readily broken under strong acidic or alkaline conditions. The hydrolysis of the ester or ether linkages between the proteins and polysaccharides tends to result in the dissolution of more protein within the extraction fluid. In view of this, it has been generally observed that as the acidity of the extraction fluid increases the protein yield from the extraction processes tends to increase. This relationship of increased yield has also been observed with increasingly alkaline solutions. That said, it has also been observed that the increase in protein yield tends to be greater with increasing acidity than with increasing alkalinity. Disadvantageously, it has been observed that strong alkaline conditions (e.g., pH greater than about 12) have a tendency to cause severe hydrolysis of the proteins, especially at temperatures above 50° C., thereby resulting in the extracted proteins having lower molecular weights and lower viscosities, which tends result in more of the extracted proteins being dissolved when washed in water following extraction, which results in decrease yield. Attempting to counter or avoid excessive hydrolysis by utilizing weak acidic (e.g., pH of about 2 to about 5) or weak alkaline conditions (e.g., pH of about 8 to about 11) results in a trade-off—in general, there is relatively less breakage of the disulfide bonds and other chemical and physical interactions between the protein constituents but significantly less protein is tends to be over a particular duration.

In view of the foregoing and experimental results to date, it has been determined that the protein extraction fluid of the present invention preferably has a pH that is no greater than about 8, more preferably less than about 7, and still more preferably less than about 5. Even more preferably, the pH of the extraction solution is at least about 1 and less than about 3. In particular, in a pH of about 2 was found to result in a particularly desirable compromise among the various parameters and qualities such as the quality or physical attributes of the extracted proteins, the protein yield, process efficiency, cost, etc. In contrast, it has been observed when processing DDGS that if it is desirable to increase yield while still obtaining relatively good quality extracted proteins the pH of the extraction fluid may be selected so that is in the range of at least about 1 and no greater than about 2. That said, it is often more important to focus on the quality of the extracted protein (e.g., molecular weight, viscosity, color, etc.) over yield and a pH of at least about 2 tends to do this but exceeding a pH of about 3 tends to result in what would generally be considered to be a commercially undesirable decrease in the yield for what would be commercially reasonable treatment durations.

As indicated above, the protein extraction fluid may also comprise a protein reducing agent. If present, the concentration of the reducing agent is preferably at least about a 0.001 weight-weight percentage (% (w/w)) of the protein extraction solution and preferably no greater than about 50% (w/w). Although not required, the reducing agent is typically added to the protein extraction fluid after the pH has been adjusted and immediately before contacting the plant-based protein source with the protein extraction fluid. This is because reducing agents tend to lose their activity upon exposure to oxygen. The addition of a reducing agent typically increases the pH of the protein extraction fluid. Advantageously, reducing activity of reducing agents typically is enhanced as pH is lowered such as at the above-described pH values for the protein reduction fluid.

Exemplary reducing agents for inclusion in the protein extraction fluid include sodium sulfite, dithiothreitol, mercaptoethanol, cysteine, sodium bisulfite, and combinations thereof. Without being limited by theory, it is believed that sodium sulfite, for example, converts into sulfurous acid during extraction and breaks the disulfide bonds in the proteins and thereby increases the solubility of the proteins in, for example, ethanol solutions. That said, experimental results to date indicate that, for practical application, there may be rough minimum and maximum threshold guideline concentrations for sulfite ions. In particular, if the concentration of sulfite ions is too low (e.g., about 0.01%) the disulfide bonds in the proteins tend not to be broken in a significant quantity and, as such, there is little, if any, effect on the protein yield. On the other hand, if the concentration of sulfite ions is too high (e.g., about 5%) the degree of disulfide bond breakage tends to be increased to a point that the molecular weights of the extracted protein(s) may be reduced to undesirable levels. Since some of the lower molecular weight proteins tend to be removed during washing and protein purification, this too can reduce the protein yield.

As with the above described oil-pigment extraction fluid, the protein extraction fluid may be contacted with the plant-based protein sourced by any appropriate method such as by spraying, soaking, immersion, etc. without our without agitation of solid material. To date, acceptable results have been obtained by contacting the solids with the protein extraction fluid in a manner so that a mixture having a liquid-to-solid weight ratio of at least about 1:1 is formed. Preferably, the liquid-to-solid ratio is no greater than about 12:1 or even 10:1. The order of addition (i.e., whether the protein extraction fluid is added to the solids or the solids is added to the fluid) tends to be irrelevant except at low solution-to-solid ratios where the fluid is preferably added to the solids to ensure even mixing/contact.

The mixture is typically kept within the temperature range of at least about 10° C. and no greater than the boiling point of the protein extraction fluid. Also, the solids and liquid are typically in contact for a duration to sufficient to remove at least some of the protein from the plant-based protein source. As is evident to one skilled in the art, the extraction system/process should be considered when selecting a temperature for protein extraction or vice versa. In general, increasing the temperature of the system, which includes the protein extraction fluid and/or the solids) tends to increase the amount of proteins that are extracted. Without being held to a particular theory, it is believed that the increase energy introduced into the system by increasing the temperature tends to break more disulfide bonds, which decreases the moleculare weights of the proteins and enhances their solubility. But it is also believed that relatively high temperatures may denature or change the structure of proteins and therefore reduce the amount of non-denatured proteins compared to what may be obtained by extracting at lower temperatures. In general, it is believed that the desired molecular weight of the extracted proteins should be favored over yield when selecting a protein extraction temperature(s).

The duration of the extraction may also vary depending on the extraction system and the temperature of extraction, but a suitable duration may be determined by one skilled in the art. For example, it is generally desirable to select a duration that, in combination with the other process parameters, allows for extraction of at least about 20 percent of the protein that is within the plant-based protein source. Typically, the plant-based protein source and the protein extraction fluid are in contact for a period that is at least about 5 minutes. Although there is essentially no required maximum duration (e.g., it could be conducted for 30 days), practical considerations would typically limit the duration to a few hours. Results to date indicate that satisfactory extraction results may be accomplished by contacting the fluid and the solids for a duration that is at least about 10 minutes and no longer than about 120 min.

After completion of the protein extraction operation, a solids portion comprising the cereal grain material and a liquid portion comprising the extraction fluid and dissolved protein(s) are separated from each other by any appropriate method(s) such as filtration and/or centrifugation. The dissolved protein(s) may be separated from the liquid portion by any appropriate method such as evaporation, filtration, centrifugation, chromatography, or any combination thereof. For example, when using ethanol as the organic solvent of the protein extraction fluid, separation has bee accomplished by centrifugation to separate the plant-based protein source from the extraction fluid comprising dissolved proteins, e.g., at 10,000 rpm for 5 min. Also, the ethanol solution containing the dissolved proteins is recovered and vaporized under low pressure at 40° C. to separate the proteins.

Characteristics of the proteins may be assessed to determine the quality and yield. The quality of the proteins is typically correlated with their molecular weight, which can be expressed using intrinsic viscosity. In general, the higher the intrinsic viscosity of the extracted proteins, the better their quality for most applications. In view of this, the process parameters of the present invention are preferably selected and/or controlled so that the intrinsic viscosity of the extracted proteins (after being separated from the extraction fluid and washed) is at least about 3 mL/g in 70% w/w aqueous ethanol solution. Preferably, the process is controlled so that at least about 80 percent of the extracted proteins have an intrinsic viscosity that is at least about 10 mL/g. Alternatively, the process is preferably controlled so that at least about 20 percent of the extracted proteins have an intrinsic viscosity that is at least about 35 mL/g.

The quality of the extracted proteins may also be analyzed by methods known in the art to determine the molecular weight of proteins, including SDS-PAGE, mass spectroscopy, chromatography, centrifugation, and filtration. The molecular weight of the extracted protein is at least about the molecular weight predicted from the protein polypeptide. In certain applications, e.g., fiber and film production, the presence of larger molecular weight species is preferred. Molecular weight species of extracted proteins larger than the predicted molecular weight of the protein polypeptide typically indicates the existence of chemical interactions among the proteins or between the proteins and other chemical moieties, e.g., carbohydrates, lipids, etc. The chemical modifications that may be present natively in the cereal grain, e.g., phosphorylation, or introduced into a byproduct during processing of the cereal grain. Additionally, chemical interactions may also be formed or deformed during zein extraction and purification, e.g., disulfide, amide, and ester bonds.

Often, a desirable quality for proteins extracted from plant-based sources is a lack of color because colorless proteins are more versatile in applications. In plant-based protein sources with pigments, it is generally desirable to decrease the intensity of the color as much as possible without affecting the properties of the proteins. Any method known in the art may be used to quantify the pigmentation, e.g., the “CIE yellowness index” of the Commission Internationale de L'Eclairage (International Commission on illumination). For use in fibers, films, and paints, the CIE yellowness index for zein is preferably within the range of about 0 to about 70 or less and more preferably within the range of about 0 to about 30. Proteins extracted in accordance with certain embodiments may suitable for such applications. For example, zein obtained by an embodiment of the present invention that includes the oil-pigment extraction operation is expected to have a CIE yellowness index of about 50. In comparison, the CIE yellowness index for zein extracted without using the oil-pigment extraction operation is expected to be about 150.

Typically, the yield of the protein is also an important concern in commercial and industrial applications. Typically, at least about 3% (w/w) of the proteins are extracted from plant-based protein sources using the method of the present invention. To be clear, reference to percentages of protein extracted or yield are based on the extracted protein in comparison to the weight of the plant-based protein source from which the protein was extracted. As mentioned above, there tends to be an inverse relationship between yield and protein quality (e.g., viscosity and/or molecular weight), therefore, process parameters are typically selected and/or controlled to result in a desired compromise between the two. Typically, the process parameters have been selected so that the yield is, in order of increasing preference, at least about 10%, 20%, 30%, or 40% (w/w). Preferably, the process parameter are selected and/or controlled so that about 45% (w/w) of the proteins are extracted from plant-based protein sources using these methods.

In addition to proteins, the liquid portion may also comprise carbohydrates, lipid complex moieties. Also, it should be noted that certain of the extracted protein may not be desired (e.g., glutelin). These undesirable or impurity constituents of the liquid portion tend to be intermixed with the desired extracted proteins upon separation from the extraction fluid. That said proteins (e.g., zein) extracted by the method of the present invention tend to have similar levels of these impurities as proteins extracted by wet milling, which contained about 80-85% protein, 15-20% lipids, and less than 0.25% starch (Parris, N.; Dickey, L. C. Extraction and solubility characteristics of zein proteins from dry-milled corn. J. Agric. Food Chem. 2001, 49, 3757-3760). The impurities may be taken into account to more precisely determine the yield of desired extracted protein(s). For example, the purity of the extracted protein may be determined by measuring the nitrogen content of the extracted fraction according to standard test methods. The protein content for extracted solids separated from the extraction fluid as determined by measuring nitrogen contents is preferably at least about 80% (w/w) and more preferably at least about 90% (w/w) of the solids fraction extracted from a plant-based protein source.

The phosphorus content of the extracted proteins may also be determined. Phosphorous in proteins imparts flame resistance, which may be important for various applications such as flame retardant fibers, paints, and films. The phosphorous content may be measured according to the Bray and Kurtz method (Bray, R. H.; Kurtz, L. T. Determination of total organic and available phosphorus in soils. Soil Sci., 1945, 59, 39-45). Based on experimental results to date, zein extracted in accordance with the present invention contained phosphorous at a concentration that is from about 0.05 to 0.08%.

In general, it has been observed that the method of present invention tends to offer one or more of the following benefits over methods know in the prior art: the yield of desirable proteins is greater and the extracted proteins tend to have higher molecular weights and less color. Because of the enhanced yield, the cost of the these extracted proteins may be lesser and because of their characteristics, they tend to be appropriate for producing high quality film, paints, adhesives, fibers, and other applications.

### EXAMPLES

The following examples illustrate the methods of extracting plant-based proteins from a cereal grain source using acidic conditions. The examples demonstrate certain methods and are not intended to limit the scope of the present invention. Those of skill in the art should, in light of the present disclosure, appreciate that many changes can be made in the specific embodiments which are disclosed and still obtain a like or similar result without departing from the spirit and scope of the invention.

### Example 1

### Extracting Protein from DDGS

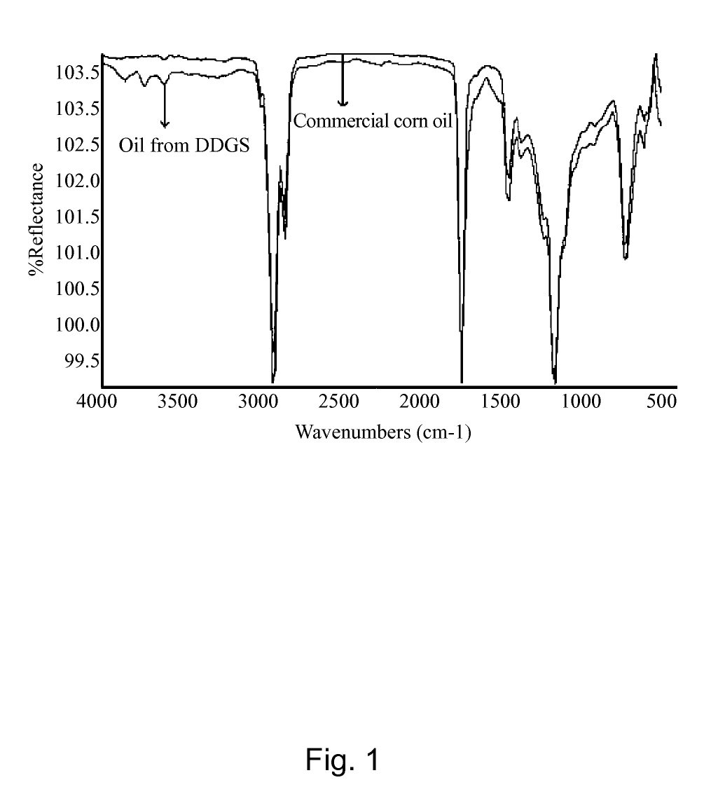

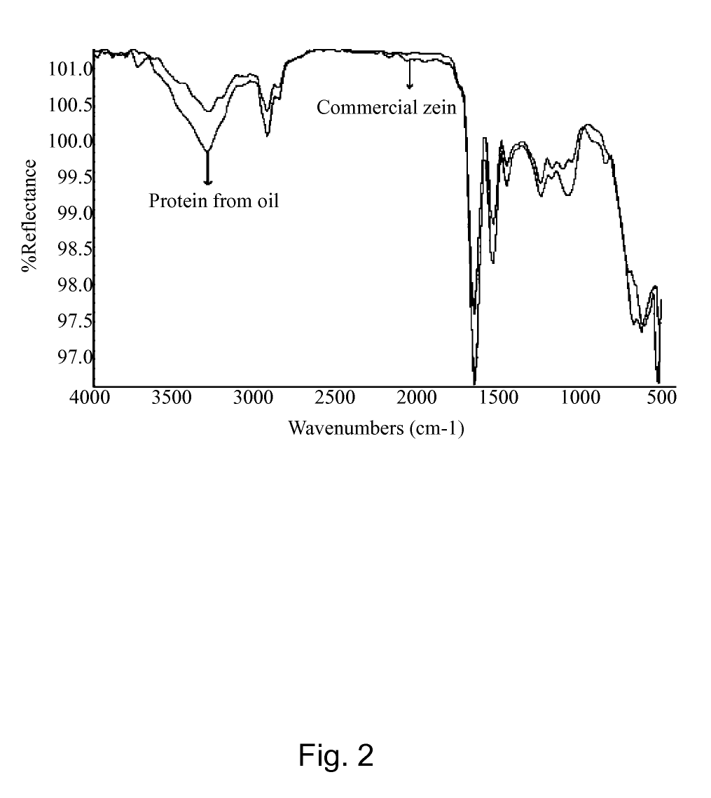

DDGS (Abengoa BioEnergy Corporation, York, Nebr.) was obtained as a byproduct of processing corn for ethanol contains 25% (w/w) protein and was yellow in color. The DDGS was powdered in a laboratory scale Wiley mill to pass through a 20 mesh dispenser. The oil in the powdered DDGS was extracted using anhydrous ethanol in a Soxhlet extraction apparatus until the extracted liquids were colorless. The oil suspension was centrifuged to separate the components after vaporizing the ethanol. Some of the proteins in DDGS were also extracted with the oil. These proteins were collected by vaporizing the anhydrous ethanol at 40° C. under low pressure. The precipitate collected was centrifuged at 10,000 rpm for 20 min to obtain the proteins. The proteins obtained were dried and weighed. About 17% (w/w) crude oil based on the dry weight of DDGS and about 1% (w/w) solids were obtained with the oil during the first extraction step. The oil obtained from DDGS has similar composition to that of corn oil available on the market, as seen from the FTIR spectrum in FIG. 1. The FTIR spectra of the extracted oils were obtained on an FTIR (model Nicolet 380; Thermo Electron Corporation). Sixty-four scans were recorded for each sample at a resolution of 32 cm⁻¹ in the reflectance mode. The solids extracted with the oil are mostly proteins and have similar spectrum to the commercial zein as seen from FIG. 2.

After extracting the oil and pigment in the first step, DDGS (˜20 g) was treated with a 70% (w/w) aqueous ethanol solution (˜200 g) at boiling temperature for 2 hours. All concentrations of the chemicals used were based on the total weight of the extracted liquids. The pH of the solution was adjusted at the beginning of the extraction using 20% (v/v) hydrochloric acid (VWR International, Bristol, Conn.) or 20% (w/w) aqueous sodium hydroxide because the pH of the solution will change gradually after the gradual dissolution of the reducing agent, such as sodium sulfite, with increasing temperatures. The solution was heated to the desired temperature in about 10 min. After the extraction, the solution was centrifuged at 10,000 rpm for 5 min to separate the alcohol solution from the other components in DDGS. The ethanol solution containing the dissolved proteins was then vaporized under low pressure at 40° C. to obtain the proteins. The collected proteins were washed in distilled water, centrifuged at 10,000 rpm for 5 min, dried, and weighed. All experiments were repeated at least twice, and the averages and error bars with ±1 standard deviations are reported for all experiments.

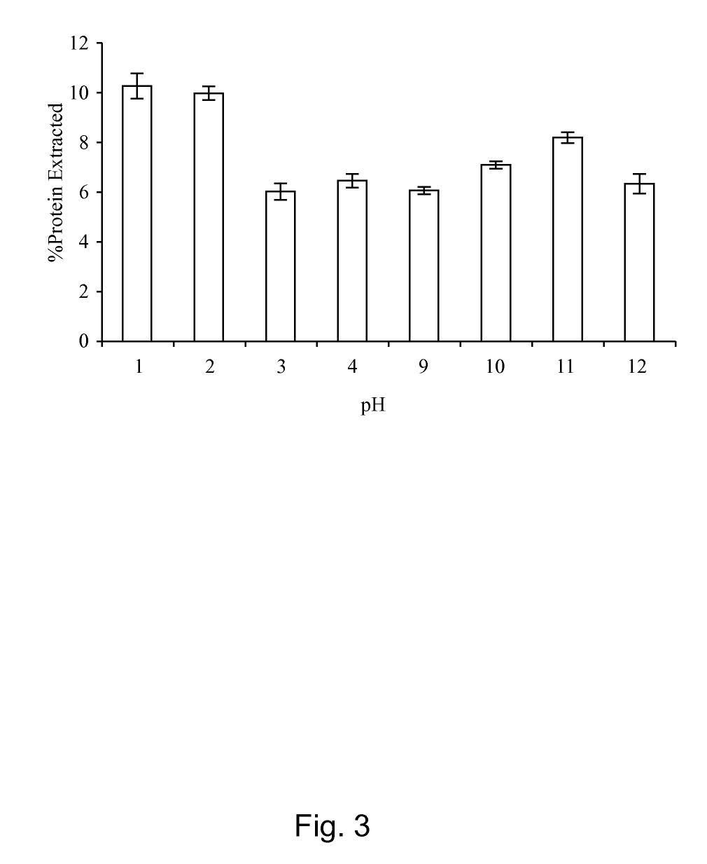

As depicted in FIG. 3, the amount of zein extracted from DDGS depended on the pH. Under highly acidic conditions (pH 1 and pH 2) about 10% (w/w) zein was obtained. Under a highly alkaline condition (pH 11) the yield was about 8% (w/w) zein. The difference between the treatments at pH 2 and pH 11 was statistically significant (p value<0.0001). The yield of zein obtained under weak acidic or weak alkaline conditions (i.e., pH 3-10) was significantly lower (in the range of 6-7% (w/w) zein). Based on the conditions used in this study, a pH of 1-2 was most suitable for obtaining a high yield of proteins from DDGS.

The intrinsic viscosities of the proteins obtained from DDGS under various conditions were determined according to ASTM standard D 2857 at a temperature of 25.0±0.1° C., and compared to that of commercially available fiber-grade zein (Freeman Industries LLC, Tuckahoe, N.Y.). “Freeman zein” or “commercial zein”, as used herein, refers to a zein (F4000) having a molecular weight of 35 kD (http://www.freemanllc.com/zein4000.html; accessed Mar., 25, 2007). The zein obtained at pH 1 had an intrinsic viscosity of about 9.6 mL/g compared to a viscosity of 31.5 mL/g for the zein obtained at pH 2 and a viscosity of 25.8 mL/g for the commercial zein. When producing zein-based fibers and films, viscosity of the protein plays an important role in their strength, elongation, and flexibility and flexibility of the zein products such as fibers and films. In view of the higher viscosity of the proteins extracted at pH 2, it would usually be considered a more desirable than pH 1, which had a higher yield. Similarly, because proteins tend to be hydrolyzed more readily under strong alkaline conditions, the commercial zein, which was extracted under alkaline conditions, had lower viscosities than the zein obtained from DDGS at pH 2.

### Example 2

### Effect of Sodium Sulfite

The effect of the addition of various concentrations of sodium sulfite on and acidic extraction process was determined. After extraction of the oil and pigment, DDGS (˜20 g) was treated with a 70% (w/w) aqueous ethanol solution (˜200 g) in the presence of a specified quantity of anhydrous sodium sulfite (98% ACS grade; VWR International, Bristol, Conn.). The pH of the solution was adjusted using 20% (v/v) hydrochloric acid (VWR International, Bristol, Conn.) or 20% (w/w) aqueous sodium hydroxide at the beginning of the extraction before adding sodium sulfite because sodium sulfite does not completely dissolve in 70% (w/w) ethanol at room temperature. The solution was heated to the desired temperature in about 10 min. After the extraction, the solution was centrifuged at 10,000 rpm for 5 min to separate the alcohol solution from the other components in DDGS. The ethanol solution containing the dissolved proteins was then vaporized under low pressure at 40° C. to obtain the proteins. The collected proteins were washed in distilled water, centrifuged at 10,000 rpm for 5 min, dried, and weighed. All experiments were repeated at least twice, and the averages and error bars with ±1 standard deviations are reported for all experiments.

About 5% of zein was extracted from the DDGS without adding any sodium sulfite but adding sodium sulfite up to a concentration of about 2.5% (w/w) increased the amounts of zein obtained. Above a concentration of about 2.5% (w/w) the amount of zein extracted decreased as seen from FIG. 3. A sodium sulfite concentration of 2.5% gave the highest yield of zein as seen from FIG. 3.

### Example 3

### Effect of Temperature

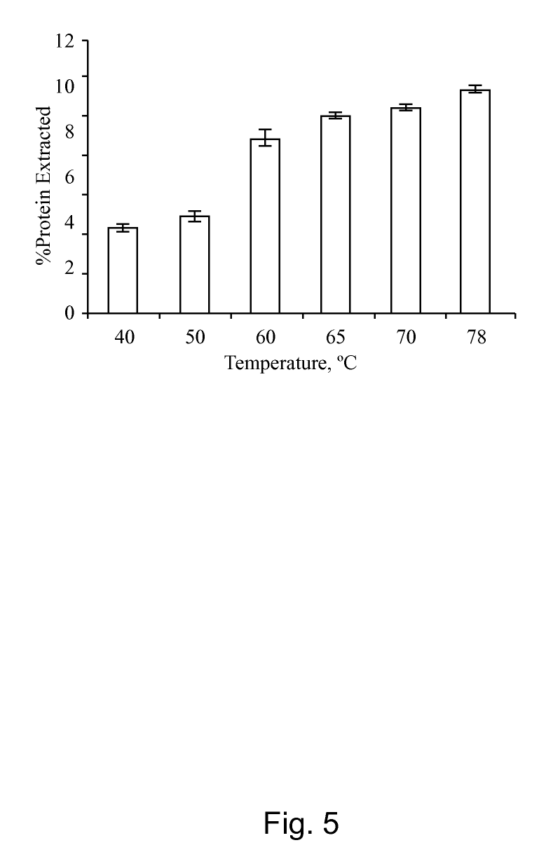

The temperature of extraction was also varied to evaluate its effect on the quality and yield of the extracted protein. Increasing the temperature of extraction increased the yield of the extractives as shown in FIG. 5. Interestingly, the yield of the extracted zein was similar at 40 and 50° C. (p value=0.1623), but there was nearly a one-fold increase in yield (8% w/w zein) when the temperature was increased from 50 to 60° C. The highest yield was obtained at the boiling temperature of ethanol solution, 78° C. The treatment at 78° C. was significantly different than the treatments at 40°, 50°, 60°, 65°, and 70° C., (p values<0.0001, <0.0001, <0.0001, <0.0292, and <0.0490, respectively).

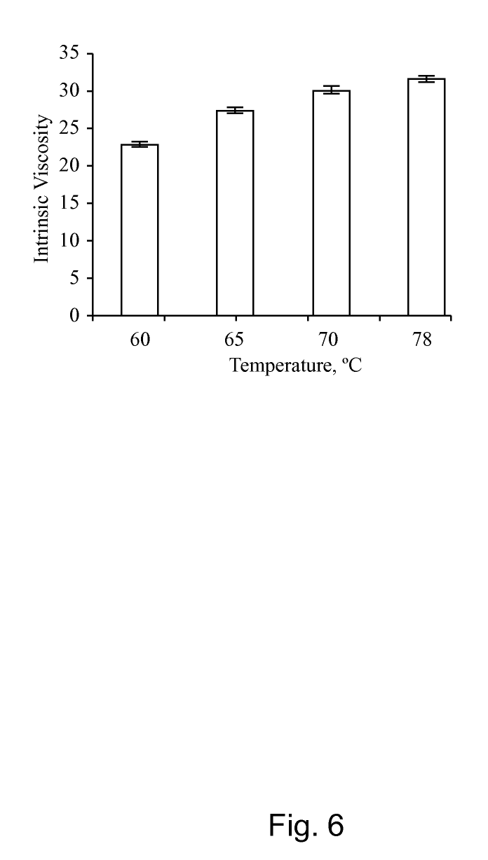

The intrinsic viscosity of the proteins obtained at various temperatures is shown in FIG. 6. The range of the intrinsic viscosity for extracted zein fell within the range of about 20 mL/g to about 32 mL/g. Increasing the temperature of extraction not only increased the yield but also the intrinsic viscosity of the protein, indicating an increase in the average molecular weight. This supports the hypothesis that relatively low temperatures are not conducive to the breaking of disulfide bonds and other molecular interactions in some of the higher molecular weight proteins and between the proteins and other components. On the basis of the yield and viscosity of the zein obtained, a temperature of about 78° C. appeared to be the optimum temperature for extraction under those conditions.

### Example 4

### Effect of Holding Time

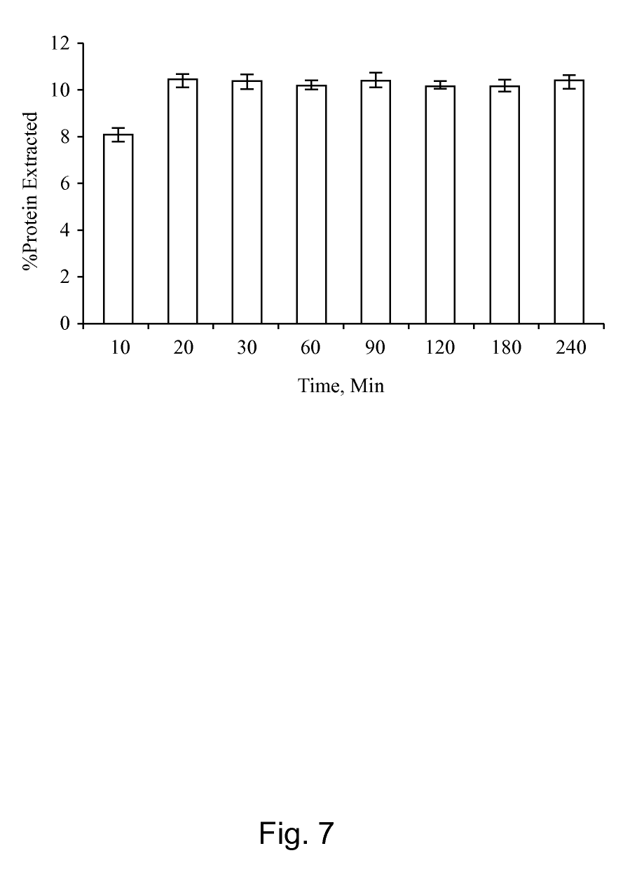

The holding time of the extraction was also tested to determine if it affected the quality and yield of the extracted protein. FIG. 7 depicts the effect of increasing extraction time on the percentage of extractives obtained. The time shown here is the holding time after the solution has reached boil from room temperature (usually in about 10 minutes). As seen from FIG. 7, about 8% yield was obtained just after 10 minutes of holding at boil and there was a significant increase in yield to about 10% after 20 minutes of boiling (p value<0.0001). There was no further significant increase in yield from 20 min to 4 h, (p values=0.8981-1.0000). The high yield of zein obtained at relatively short extraction times compared to the extraction times used in previous known methods may be due to the low pH and higher temperature used in this research. For example, one previously used method for extracting crude zein from DDGS involved three steps of 30 min each at 60° C. at a pH of 10. The amount of pure zein obtained by that known method was (˜3.3% w/w based on weight of DDGS used), much lower than the yield of zein obtained in this study.

### Example 5

### Effect of Solvent to Solids Ratio

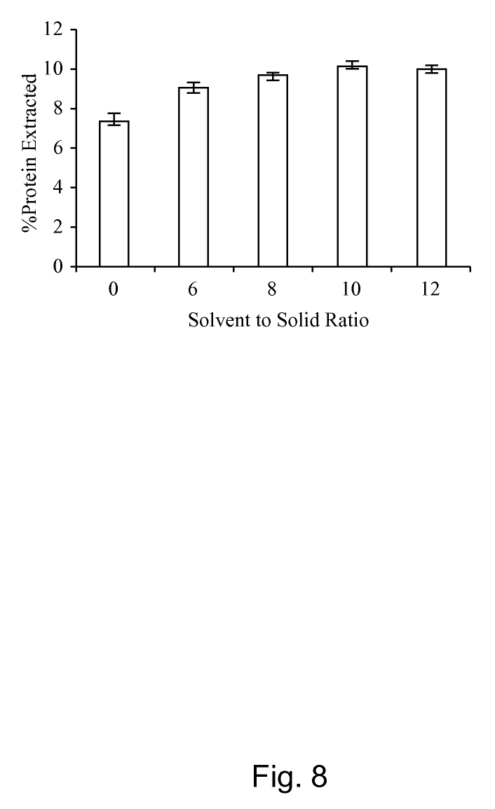

The solvent to DDGS ratio of the extraction was also tested to determine if it affected the quality and yield of the extracted protein. As depicted in FIG. 8, the solvent-to-DDGS ratio within the range of about 6:1 to about 12:1 and produced relatively high yields of zein when using a sodium sulfite concentration of 0.25%. Without being held to a particular theory, it is believed that the decrease in dissolved zein seen with low solvent-to-solid ratios such as 4:1 is due to limited availability of the solvent. Varying the solvent-to-solids ratios gave yields with statistically significant differences, with p values of <0.0001 between 4:1 and 6:1, <0.0107 between 6:1 and 8:1, and <0.0204 between 8:1 and 10:1. But the yield obtained with solids-to-solvents ratio between 10:1 and 12:1 did not produce significant differences with a p value of 0.7467. The yield of extracted protein may also decrease at very high ratios, because some of the proteins, especially the proteins with lower molecular weights, tend to be dissolved easily and are removed during purification.

### Example 6

### Analysis of Extracted Zein

The extraction conditions such as the pH, concentration of reducing agent, time, and temperature were controlled to obtain zein with higher yield, less color, and better quality than previously reported. For example, the yield of the zein was a relatively high about 44% (w/w) of the total proteins in the DDGS.

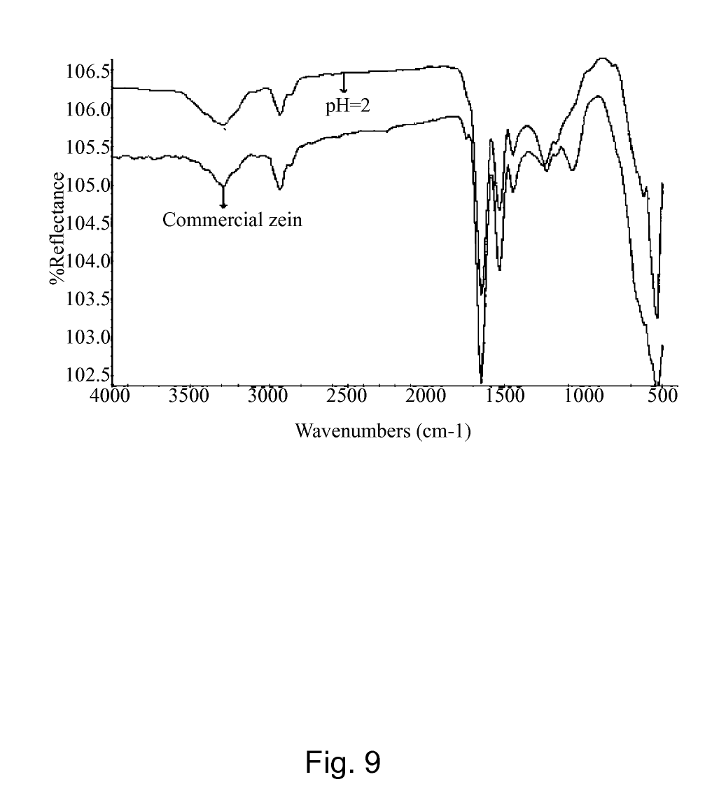

Compositional analysis and FTIR analysis was also performed on the proteins obtained from DDGS. The extractives obtained from DDGS at various pH conditions have similar compositions to that of Freeman zein as determined by compositional analysis and FTIR analysis. The nitrogen content of commercial zein and zein extracted from DDGS was analyzed using the Dumas method (Elementar Rapid N) (Bremner, “Nitrogen-Total.” Sparks, D. L., Ed.; In Methods of Soil Analysis, Part 3: Chemical Methods; Soil Science Society of America, Inc.: Madison, Wis., 1996; No. 5, pp. 1085-1089). The protein contents of the two types of extracted protein were calculated by multiplying the nitrogen content by a factor of 6.5. The protein samples were made into pellets and used to obtain the infrared spectrum as seen from the FTIR spectrum in FIG. 2. The FTIR spectra of the protein pellets were obtained on an FTIR (model Nicolet 380; Thermo Electron Corporation). Sixty-four scans were recorded for each sample at a resolution of 32 cm⁻¹ in the reflectance mode. The spectra of zein obtained under all pH conditions were similar; only the spectra determined for pH 2 is shown in FIG. 9. The major difference in the peaks of zein obtained in this experiment and that of Freeman zein is the presence of a sharp peak at about 1100 cm⁻¹. This peak at 1100 cm⁻¹ is from the phosphorus present in the protein as a phosphoryl group (CRC Handbook of Chemistry and Physics, 82nd ed.; Lide, D. R., Ed.; CRC Press LLC: New York, N.Y., 2001; pp 9-89). In particular, Freeman zein has a phosphorous content of 0.02% compared to 0.08% in the zein obtained from DDGS according to this invention (the content is reported as % w/w based on the dry weight of zein used). The phosphorus content in the zein extracted from DDGS was determined according to the method of Bray and Kurtz (Bray and Kurtz, “Determination of total organic and available phosphorus in soils.” Soil Sci. 1945, 59, 39-45).

SDS-PAGE analysis was performed to assess the quality of the zein extracted from DDGS by comparing the electrophoresis patterns between the zein extracted from DDGS and commercial zein. SDS-PAGE electrophoresis was performed on the samples with and without reduction. To observe the electrophoretic patterns in the reduced state, about 10 mg of commercial zein and proteins extracted from DDGS were powdered and mixed with 200 μL of SDS-PAGE 1× sample buffer (0.83 mM Tris-HCl, 2% SDS, 2% β-mercaptoethanol, 10% glycerol, and double-distilled water). The unreduced samples were prepared using about 10 mg of commercial zein and proteins extracted from DDGS. The samples were powdered and mixed with 200 μL of SDS-PAGE 1× sample buffer without reducing agent (0.83 mM Tris-HCl, 2% SDS, 10% glycerol, and double-distilled water). The four samples were kept at room temperature for 20 min. The samples were then centrifuged and the clear top layer was collected and the protein concentrations in the solution were measured (BioRad protein assay). The samples were then diluted to a concentration of 40 μg of proteins per 30 μL of the solution using a 1× reducing and non-reducing buffer for each sample of the commercial zein and the zein obtained from DDGS. Each sample was heated for 1 min at boil and then loaded, one sample per lane, in the gel. After electrophoresis, the gel was washed twice and stained with Coomassie Brilliant Blue G-250. After standing overnight, the gel was flushed with deionized water and put in a destained liquid until a clear background was formed. The molecular weights of the proteins were determined by comparison to a standard mixture of marker proteins ranging in molecular weight from 10 to 250 kD (BioRad Chemical Co.)

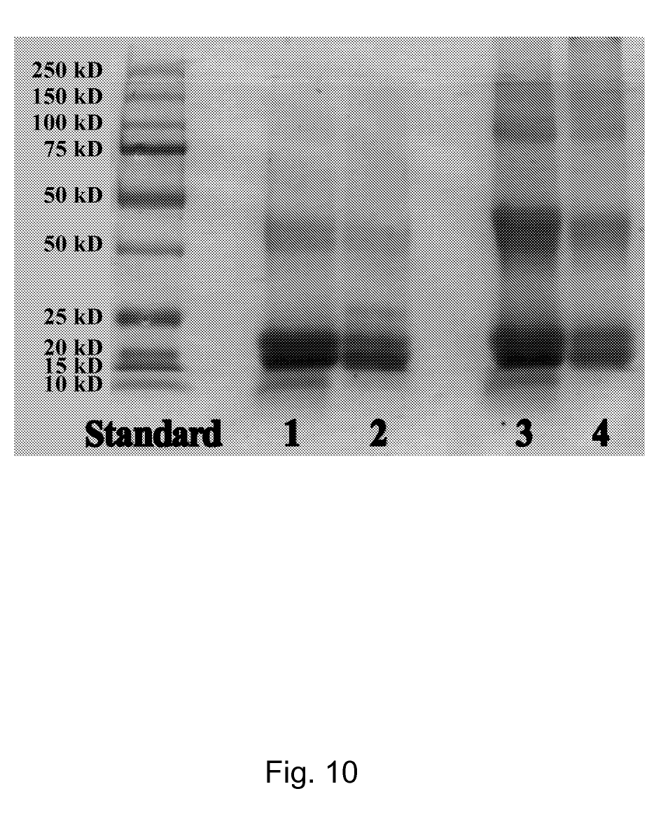

FIG. 10 shows the results of the SDS-PAGE analysis. Lanes 1 and 2 in the gel are sample in reduced form of the commercial zein and zein obtained form DDGS, respectively, and lanes 3 and 4 are samples in unreduced form of the commercial zein and zein obtained form DDGS, respectively. As seen from FIG. 10, the unreduced commercial zein (Lane 3) and zein from DDGS (Lane 4) have similar molecular weight bands in the 15-150 kD range. Additionally, the test showed that the DDGS zein has some higher molecular weight proteins (>250 kD), whereas the commercial zein has lower molecular weight proteins (<10 kD). Without being bound to a particular theory, it is believed that the higher molecular weight proteins in the DDGS zein may be due to the crosslinking of proteins during production of DDGS. Lower molecular weight proteins (<10 kD) were not seen in lane 4 gel (the DDGS zein) gel because they were probably removed during the ethanol fermentation process and/or defatting and washing during zein extraction. In the reduced form, the DDGS zein has some proteins around 25 kD (lane 2) not seen in the commercial zein (lane 1), whereas the commercial zein has lower-molecular-weight proteins in the 15-10 kD range that are not present in the DDGS zein. The proteins above 75 kD seen in both the commercial and DDGS zein in the unreduced form disappeared after reduction mainly due to the reduction of the disulfide bonds in the proteins. It is believed that the presence of non-disulfide bonds in the DDGS zein is most likely the main reason for the presence of some proteins around 25 kD, as seen from lane 2 in FIG. 10.

All references cited in this specification, including without limitation all journal articles, brochures, manuals, periodicals, texts, manuscripts, website publications, and any and all other publications, are hereby incorporated by reference. The discussion of the references herein is intended merely to summarize the assertions made by their authors and no admission is made that any reference constitutes prior art. Applicants reserve the right to challenge the accuracy and pertinence of the cited references.

It is to be understood that the above description is intended to be illustrative and not restrictive. Many embodiments will be apparent to those of skill in the art upon reading the above description. The scope of the invention should therefore be determined not with reference to the above description alone, but should be determined with reference to the claims and the full scope of equivalents to which such claims are entitled.

When introducing elements of the present invention or an embodiment thereof, the articles “a”, “an”, “the” and “said” are intended to mean that there are one or more of the elements. The terms “comprising”, “including” and “having” are intended to be inclusive and mean that there may be additional elements other than the listed elements. Additionally, it is to be understood an embodiment that “consists essentially of” or “consists of” specified constituents may also contain reaction products of said constituents.

The recitation of numerical ranges by endpoints includes all numbers subsumed within that range. For example, a range described as being between 1 and 5 includes 1, 1.6, 2, 2.8, 3, 3.2, 4, 4.75, and 5.

What is claimed is:

1. A method for processing a plant-based protein source, the method comprising contacting the plant-based protein source with a protein extraction fluid to dissolve protein from the plant-based protein source into the protein extraction fluid and isolating the dissolved protein from the protein extraction fluid, wherein the protein extraction fluid comprises a protein reducing component for breaking disulfide bonds between proteins and has a pH that is no greater than about 8.

2. The method of claim 1 wherein the plant-based protein source comprises a cereal grain, a cereal grain byproduct, or a combination thereof.

3. The method of claim 2 wherein the cereal grain is selected from the group consisting of corn, wheat, sorghum, canola, barley, soybean, and combinations thereof and the cereal grain byproduct is selected from the group consisting of distillers dried grains, gluten meal, gluten feed, and combinations thereof.

4. The method of claim 1 wherein the protein extraction fluid has a pH that is less than 7.

5. The method of claim 1 wherein the protein extraction fluid has a pH that is less than about 5.

6. The method of claim 4 wherein the protein extraction fluid has a pH that is at least about 1.

7. The method of claim 6 wherein the protein extraction fluid has a pH that is less than about 3.

8. The method of claim 1 wherein the protein extraction fluid is at a temperature that no greater than the boiling point of the protein extraction fluid.

9. The method of claim 1 wherein the protein reducing component is selected from the reducing agents including but not limited sodium sulfite, dithiothreitol, mercaptoethanol, cysteine, sodium bisulfite, and combinations thereof.

10. The method of claim 9 wherein the protein reducing component constitutes at least about 0.001% and no greater than about 50% by weight of the protein extraction fluid.

11. The method of claim 1 wherein the protein extraction fluid is a solution that further comprises an organic solvent component.

12. The method of claim 11 wherein the organic solvent component constitutes at least about 5% by weight of the protein extraction fluid.

13. The method of claim 11 wherein the organic solvent component is selected from the group consisting of alcohols, alkalis, ketones, amines, amides, acids, and any combination thereof.

14. The method of claim 13 wherein the organic solvent component is ethanol and it is at a concentration that is at least about 5% and no greater than about 95% by weight of the protein extraction fluid.

15. The method of claim 11 wherein the protein extraction fluid further comprises water.

16. The method of claim 15 wherein the water is at a concentration that is no greater than about 95% by weight of the protein extraction fluid.

17. The method of claim 11 wherein the protein extraction fluid further comprises a pH adjusting component.

18. The method of claim 17 wherein the pH adjusting component is selected from the group consisting of hydrochloric acid, sodium hydroxide, sodium carbonate, acetic acid, inorganic acids, organic acids, and combinations thereof.

19. The method of claim 1 wherein the plant-based protein source further comprises an oil, a pigment, or a combination thereof that are extracted from the plant-based protein source by contacting the plant-based protein source with an oil-pigment extraction fluid to dissolve oil, pigment, or both from the plant-based protein source into the oil-pigment extraction fluid before contacting the plant-based protein source with the protein extraction fluid.

20. The method of claim 19 wherein the oil-pigment extraction fluid comprises an organic solvent selected from the group consisting of an alcohol, benzene, hexane, and combinations thereof.

21. The method of claim 19 wherein the oil, pigment, or both dissolved in the oil-pigment extraction fluid is isolated from the oil-pigment extraction fluid.

22. The method of claim 19 wherein the oil-pigment extraction fluid dissolves protein from the plant-based protein source and said protein is isolated from the oil-pigment extraction fluid.

23. A method of separating oil, pigment, and protein from a cereal grain material, the method comprising:
performing a first treatment on the cereal grain material, the first treatment comprising contacting the cereal grain material with an anhydrous alcohol to form a first mixture having a liquid-to-solid weight ratio of at least about 1:1 and no greater than about 1000:1 and a temperature of at least about 10° C. and no greater than the boiling point of the anhydrous alcohol for a duration sufficient to remove substantially all of the oil, pigment, or both from the cereal grain material;
separating the first mixture into a first solids portion comprising the cereal grain material and a first liquid portion comprising anhydrous ethanol and dissolved oil, pigment, or both;
performing a second treatment on the first solids portion, the second treatment comprising contacting the cereal grain material with an acidic aqueous alcohol solution that comprises a protein reducing agent for breaking disulfide bonds between proteins to form a second mixture having a liquid-to-solid ratio of at least about 1:1 and no greater than about 1000:1 and a temperature of at least about 10° C. and no greater than about the boiling point of the acidic alcohol solution for a duration that is at least about 10 minutes and no greater than about 24 hours; and
separating the second mixture into a second solids portion comprising the cereal grain material and a second liquid portion comprising the acidic alcohol solution and dissolved protein.

24. The method of claim 23 further comprising separating the dissolved oil, pigment, or both from the first liquid portion by a method selected from the list consisting of evaporation, filtration, centrifugation, chromatography, and combinations thereof; and separating the dissolved protein from the second liquid portion by a method selected from the list consisting of evaporation, filtration, centrifugation, chromatography, and combinations thereof.

25. The method of claim 23 wherein the anhydrous alcohol is anhydrous ethanol; wherein the alcohol of the acidic aqueous alcohol solution is ethanol and it is at a concentration of at least about 5% and not greater than about 95% by weight; wherein the acidic aqueous alcohol solution further comprises hydrochloric acid; wherein the acidic aqueous alcohol solution has a pH that is at least about 1; wherein the protein reducing agent is selected from the group consisting of sodium sulfite, dithiothreitol, mercaptoethanol, cysteine, sodium bisulfite, hydroxy compounds and combinations thereof and is at a concentration such that it is at least about 0.01% and no greater than about 30% by weight of the dry weight of the cereal grain material; and wherein the cereal grain material is distillers dried grains with solubles (DDGS) and the dissolved protein is zein that has an intrinsic viscosity that is at least about 3 mL/g.
